(19) United States
(12) Patent Application Publication
(10) Pub. No.: US 2008/0249393 A1
(43) Pub. Date: Oct. 9, 2008

(54) METHOD AND APPARATUS FOR ENHANCEMENT AND QUALITY IMPROVEMENT OF ANALYTE MEASUREMENT SIGNALS

(76) Inventors: Alexander Finarov, Rehovot (IL); Yossie Kleinman, Rehovot (IL)

Correspondence Address: SMITH FROHWEIN TEMPEL GREENLEE BLAHA, LLC, Two Ravinia Drive, Suite 700, ATLANTA, GA, 30346, US

(21) Appl. No.: 11/696,715

(22) Filed: Apr. 4, 2007

(51) Int. Cl. A61B 5/1455

(52) U.S. Cl. 600/391

(57) ABSTRACT

While measuring analytes in an object, movements between the object and a device that is taking the measurement may adversely effect the accuracy of the measurements. A probe that engages an object includes a sticky surface that comes into contact with the object to alleviate the relative movement between the probe and the object. The sticky substance can be applied in a variety of manners and embodiments and in general, helps to reduce the relative movement. In addition, the probe may include the ability to apply pressure to the object. The pressure can range from a slight pressure to help reduce relative movement to a pressure that exceeds systolic pressure, thereby constricting the flow of blood through the object.

### TECHNICAL FIELD

The present device is in the field of medical instrumentation and, in particular, non-invasive measurements of physiological parameters of subjects.

### BACKGROUND

In recent years, several techniques have been proposed for non-invasive determination of physiological parameters of patients or objects, such as oxygen saturation, hemoglobin, glucose, bilirubin, cholesterol and others that collectively may be termed analytes. Among the methods frequently used are methods that utilize light and especially Red and Near Infrared (RNIR) radiation that is transmitted or reflected from a blood perfused fleshy medium. (In the text of the present disclosure, light is interpreted as electromagnetic radiation.) Usually, the radiation consists of a plurality of wavelengths selected from a broad radiation spectrum. Each analyte responds differently to different wavelengths. Analyses of absorption, scattering, transmission or reflection of different wavelengths by blood, interstitial fluids, tissue, or blood perfused fleshy medium, will all be henceforth termed radiation-object interaction products, and assist in determination of the desired analyte concentration.

The sources of RNIR radiation and the corresponding detectors are usually embedded in different shapes and configurations in a probe attached to a measurement object, which is typically the subject's finger, earlobe, or other part of the body. Both the probe and the object should be stable and maintain their relative position in the course of the measurement. Minute relative movements between the probe and subject, which occur during the measurement and are so called motion artifacts, may distort the measurement results. Complicated algorithms, such as one disclosed in U.S. Pat. No. 5,743,262 to Lepper et al., or Masimo, Inc., publication # LAB1035M (www.massimo.com), are applied to reduce the influence of motion artifacts.

In order to keep the probe in a stable relation to the object, some of the probes, e.g., disclosed in U.S. Pat. Nos. 6,461,305 and 6,488,633 to Schnall, apply a certain pressure to the object. These probes fall short in preventing relative displacement between the object and the probe. The pressure distribution by itself may be a source of additional measurement errors.

Consequently, it is desirable to have a probe, a method of using the probe for physiological parameter measurement, and an apparatus implementing such method that would be free or substantially reduce the influence of motion artifacts.

### SUMMARY

The present invention provides a solution to the above-described needs in the art, as well as other needs by, in general, providing a probe for the measurement of analytes that operates to reduce the relative motion between the object from which the measurements are being taken and the probe. Several embodiments of the present invention are presented herein and each such embodiment, although it may be a patentable invention in and of itself, is presented as a non-limiting example of the present invention. For instance, in one embodiment of the invention, a sticky material is applied in a variety of manners to either a surface of the probe that is in contact with the object, or to the object itself. In other embodiments, sticky pads or flexible pins are used to help maintain the relative position between the probe and the object.

Another aspect of the present invention is the incorporation of pressure devices into the probe. The pressure devices serve at least two purposes. One purpose is to further restrict relative movement of the object and probe. However, another purpose is to provide for the restriction or even cessation of blood flow. For instance, an occlude-release cycle mode can be created and the analyte measurements can be taken during this cycle or at strategic points in the cycle depending on the analyte being measured.

Further embodiments, aspects and features of the present invention are presented in the detailed description. It will be appreciated that not all of the aspects, features and embodiments presented are necessary elements of the invention and in fact, although various embodiments may be individually patented, the present invention is not limited to any particular set of features and/or aspects.

### BRIEF LIST OF DRAWINGS

The disclosure is provided by means of non-limiting examples only, with reference to the accompanying drawings. The drawings are not necessarily to scale, emphasis instead being placed upon illustrating the principles of the devices and methods.

FIGS. 1A and 1B are schematic illustrations of some exemplary embodiments of the probe.

FIGS. 2A-2D are schematic illustrations of a cross section of one of the exemplary embodiments of the probe.

FIG. 3 is a schematic illustration of an additional exemplary embodiment of the probe.

FIG. 4 is a schematic illustration of yet another exemplary embodiment of the probe.

FIGS. 5A-5D are schematic illustrations of further exemplary embodiments of the probe.

FIG. 6 is a schematic illustration of an exemplary method of application of the probe.

FIGS. 7A and 7B are schematic illustrations of an exemplary method of application of the probe and a pressure application article for measurement of physiological parameter.

FIG. 8 is a schematic illustration of an exemplary embodiment of the probe combined with a pressure application article.

FIGS. 9A and 9B are schematic illustrations of additional exemplary embodiments of the application of a sticky surface for measurement of physiological parameters.

FIG. 10 is a schematic illustration of a double sticky wrapping for the measurement of physiological parameter.

### DETAILED DESCRIPTION OF THE EXEMPLARY EMBODIMENTS

The method and apparatus constructed according to the method may be understood with reference to the drawings and the accompanying description, wherein like numerals of reference designate like elements throughout the text of the disclosure. In this regard, directional terminology, such as “top,” “bottom,” “front,” “back,” “upper,” “lower,” etc., is used with reference to the orientation of the Figure(s) being described. Because components of embodiments of the present probe can be positioned in a number of different orientations, the directional terminology is used for purposes of illustration and is in no way limiting.

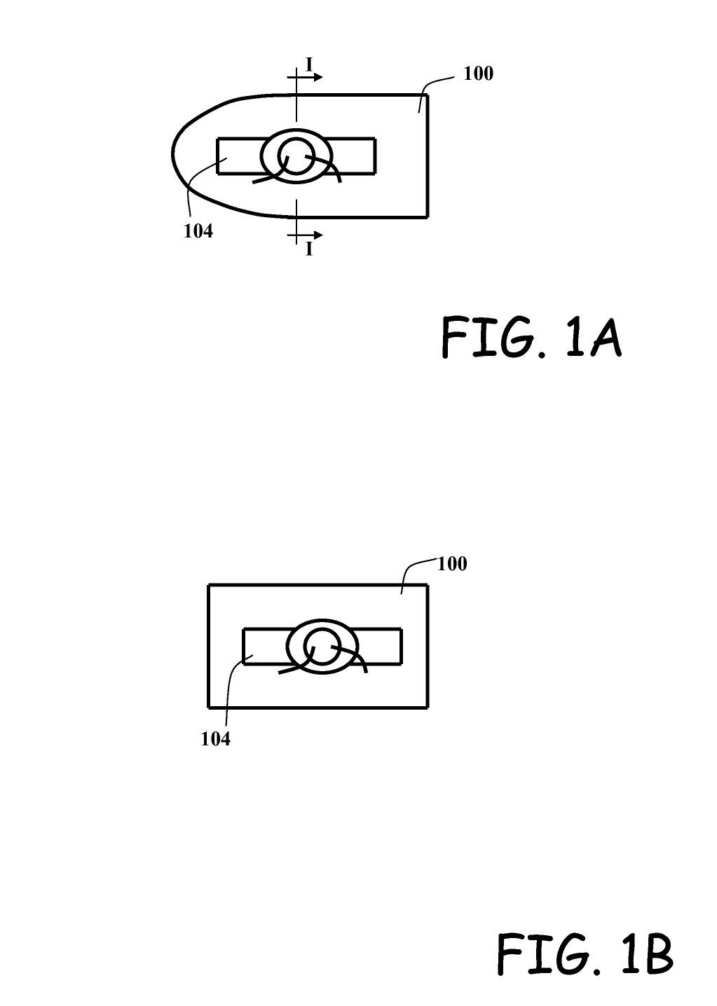

Reference is made to FIGS. 1A and 1B, which are schematic illustrations of some exemplary embodiments of the probe that may be used in non-invasive patient or object physiological parameters determination. Probe 100 has a body that may be of a thimble-like shape (FIG. 1A) or sleeve-like shape (FIG. 1B). Probe 100 may have one or more ribs 104 increasing the stiffness of the structure. Depending on the desired stiffness, ribs 104 may be located on upper and lower halves of probe 100.

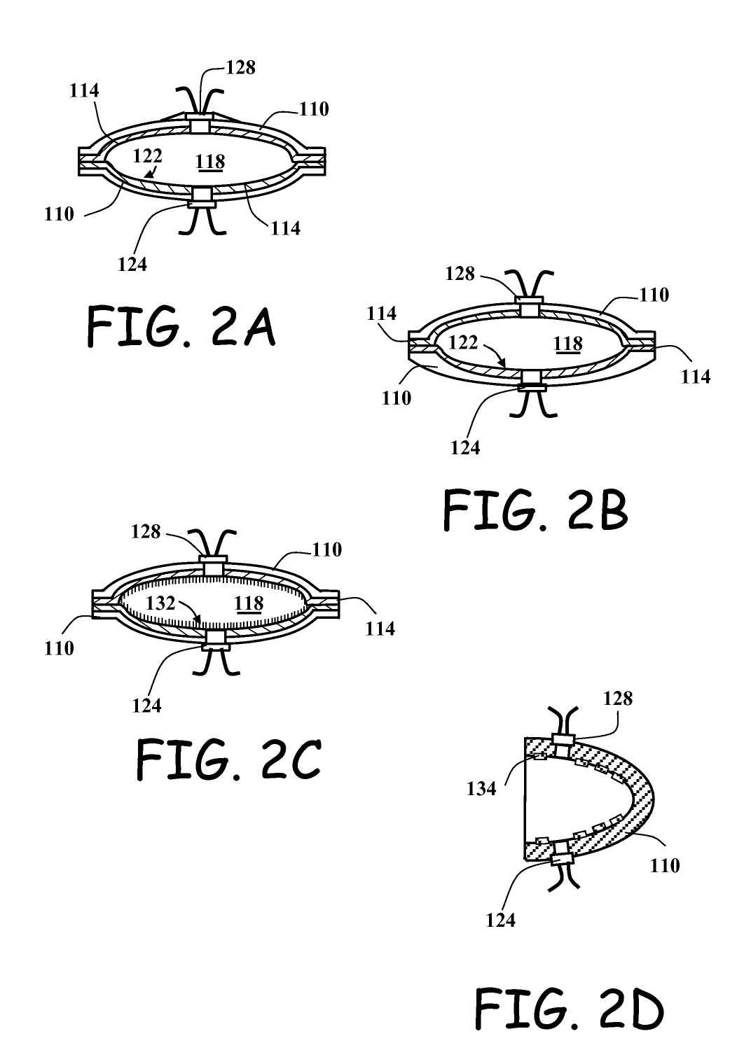

FIGS. 2A-2D are schematic illustrations of a cross section of an exemplary embodiment of the probe. The body of probe 100 is a resilient structure consisting of an outer layer 110 and inner layer 114. The body may have a symmetric form (FIG. 2A), or an asymmetric form as shown in FIG. 2B. Outer layer 110 is stiffer than inner layer 114 and it develops and supports uniform pressure on the object (not shown) when the object is inserted in the opening 118 of probe 100. Surface 122 of inner layer 114 that engages the object when it is inserted in opening 118, possess sticky properties. As will be explained below the sticky properties may be achieved by use of proper materials, material processing, or coating. They should be such that when inserted in the probe the object adheres temporarily to the sticky surface 122 and an effort is required to reposition it or cause relative movement between the object and probe 100. Because of this, the motion artifacts caused by relative movement between the probe and the object are eliminated or substantially reduced and do not affect the measurement process.

A source of radiation 124 such as an incandescent lamp, LED, Laser or VCSEL, and a suitable radiation detector 128, are built into probe 100. FIG. 2 and subsequent figures show a radiation source 124 and detector 128 locations adapted for transmission measurement. It should be clear, however, that their location may be adapted to measure reflection from the object and other radiation-object interaction products. Multiple sources and detectors measuring both the reflection and transmission may be incorporated in probe 100 as well. Typically, the sources produce radiation in a wavelength range between 400 nm and 2500 nm. Usually, at least two wavelengths are used for a measurement.

The outer layer 110 and inner layer 114 may be manufactured from silicone or similar material and joined by application of adhesive, lamination, or any other known method. The mechanical properties of the outer layer 110, however, are preferably different from the mechanical properties of the inner layer 114. For example, the hardness of the inner silicone layer could be in the range of Shorr value of 5-10, where the outer layer may have hardness values exceeding Shorr 60 or more. Silicone having hardness value of Shorr 15 and lower has a sticky surface that adheres practically to everything, although the strength of adhesion may be different among different materials and surfaces.

In an alternative embodiment, illustrated in FIG. 2C, inner layer 114 may be manufactured from silicone having a large number of protruding columns or needles 132 of few tenths of micron size. The height and elasticity of silicone columns 132 should be such as to enable fixation on the object without damaging it. In one of the embodiments, illustrated in FIG. 2D, inner layer may be made of sections 134 of sticky material. The amount of sections 134 and their surface should be sufficient to hold the object and restrict a relative movement between the object and the probe and in particular between object and radiation source 124 and detector 128. A sleeve-like probe may be implemented in a similar way.

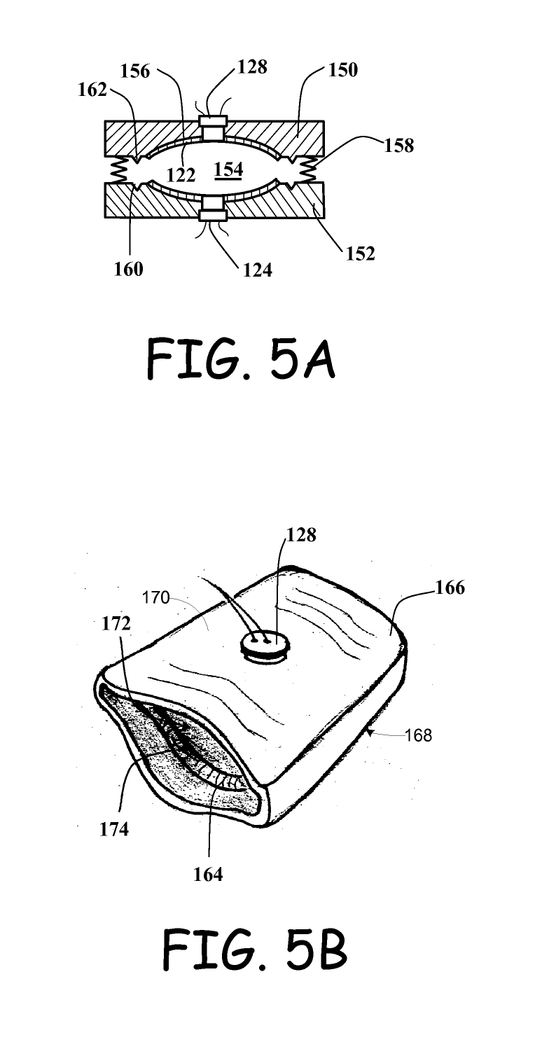

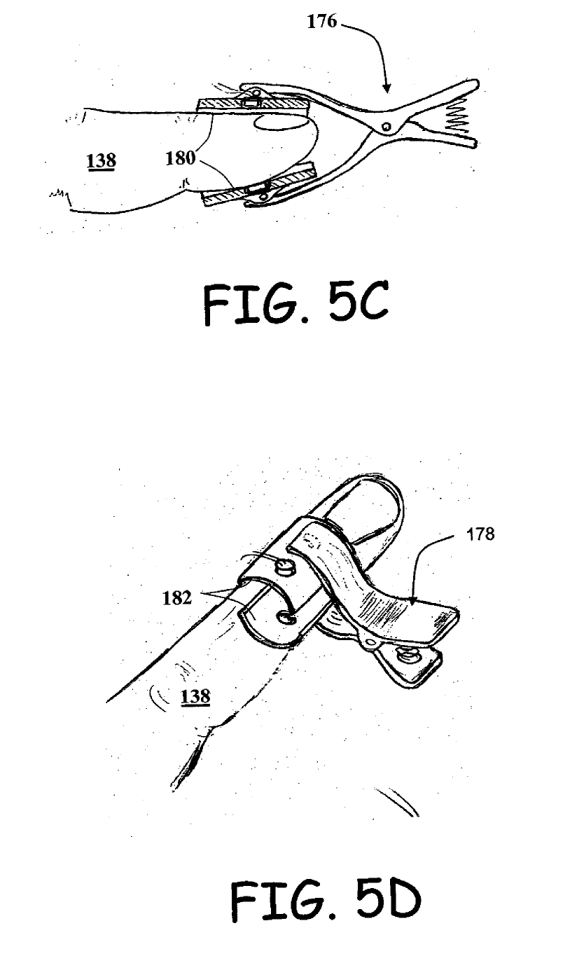

The optical signal measured by the detector 128 is a weak one and typically is affected by ambient illumination. Lightproof silicone or similar material should be used for at least one of the layers 110 or 114 of probe 100. Opening 118 that receives the object may be shielded from ambient illumination by adding light proof baffles or making a ring of porous material 164 (FIG. 5B) at the end of probe 100. Silicone columns 132 may be implemented to have a height that would allow shielding of the opening 118 that receives the object, from ambient light. Columns 132 allow free passage of air and reduce or eliminate object sweating that occurs when it is inserted into the probe 100. Alternatively, lightproof air permeable material may be used as a layer in the manufacturing of probe 100. A large selection of porous synthetic material, such as acrylic materials, Dacron, porous polyethylene, Proplast II and other similar materials may be suitable for such task.

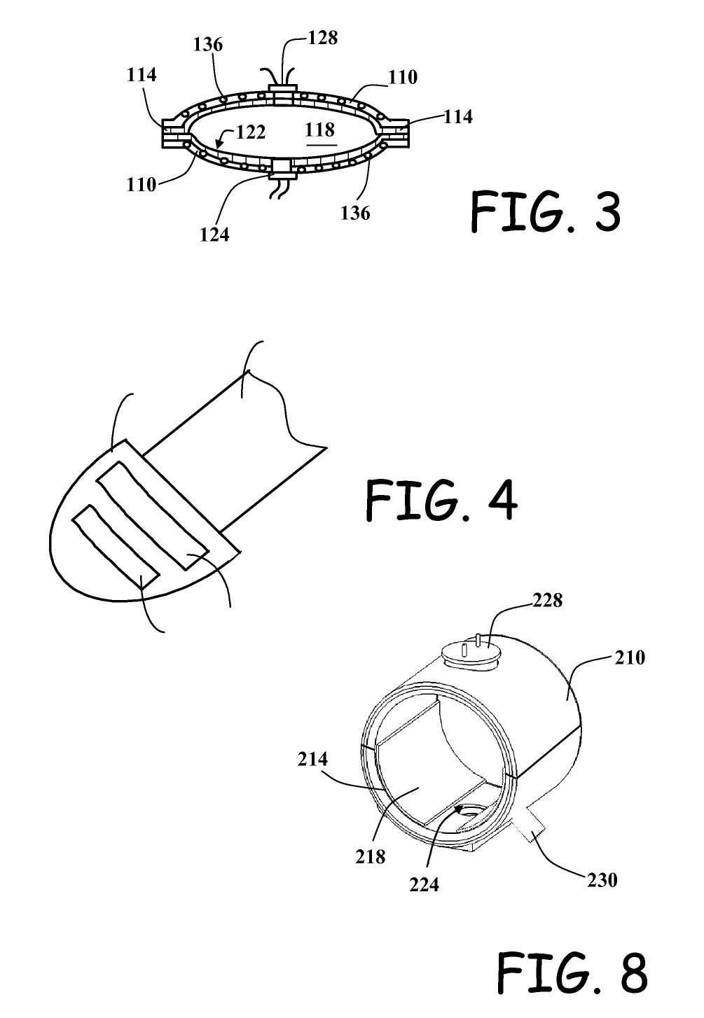

To sit firm on an object, probe 100 should develop certain pressure, generally uniformly distributed over the surface of object. FIG. 3 is a schematic illustration of an additional exemplary embodiment of the present probe. Depending on the pressure required, probe 100 may be armored by spring steel strips 136 and linear or spiral springs. Steel strips 136 may be embedded or molded together with the outer layer 110, or the inner layer 114, or located between the layers. As shown in FIG. 4, steel or plastic strips 140 or 142 may be external to probe 100. Other numerals in FIG. 4 mark the object 138 inserted in probe 100 and circumferential spring strip 140 or longitudinal springs 142. The internal or external armored elements are effective in generating uniform pressure and enhancing the movement restricting effect of the sticky surface. The size and stiffness of the armored elements may be selected such as to ensure that the pressure developed by them does not substantially affect the measurement results.

FIGS. 5A through 5D are schematic illustrations of further exemplary embodiments of the present invention. FIG. 5A illustrates a probe 148 that includes an upper 150 and a lower 152 halves and an opening 154. Linings 156 made of sticky material or strips of sticky material, or at least having a sticky surface 122 are attached to the inner surfaces of the upper and lower halves. Springs 158 ensure the desired pressure and groves 160 with protrusions 162 ensure that the opening 154 is light proof when an object is inserted into it.

FIG. 5B is a schematic illustration of probe 166 fabricated as two symmetric or asymmetric halves 168 and 170 of resilient light proof material. Probe 166 has an opening 172 that receives the measurement object (not shown). Before each measurement a sticky spray, such as Repositionable Adhesive Spray 75, commercially available from 3M, Saint Paul Minn., USA is applied such as to cover the inner surface 174 of opening 172. Upon completion of measurement, the spray may be removed by alcohol.

FIGS. 5C and 5D are schematic illustrations of additional clip-like probe embodiments. Clip-like probes 176 and 178 respectively have the surfaces 180 and 182 being in contact with the object 138, such as finger or earlobe, and are made of sticky material, or as disclosed above, coated on demand by sticky coating.

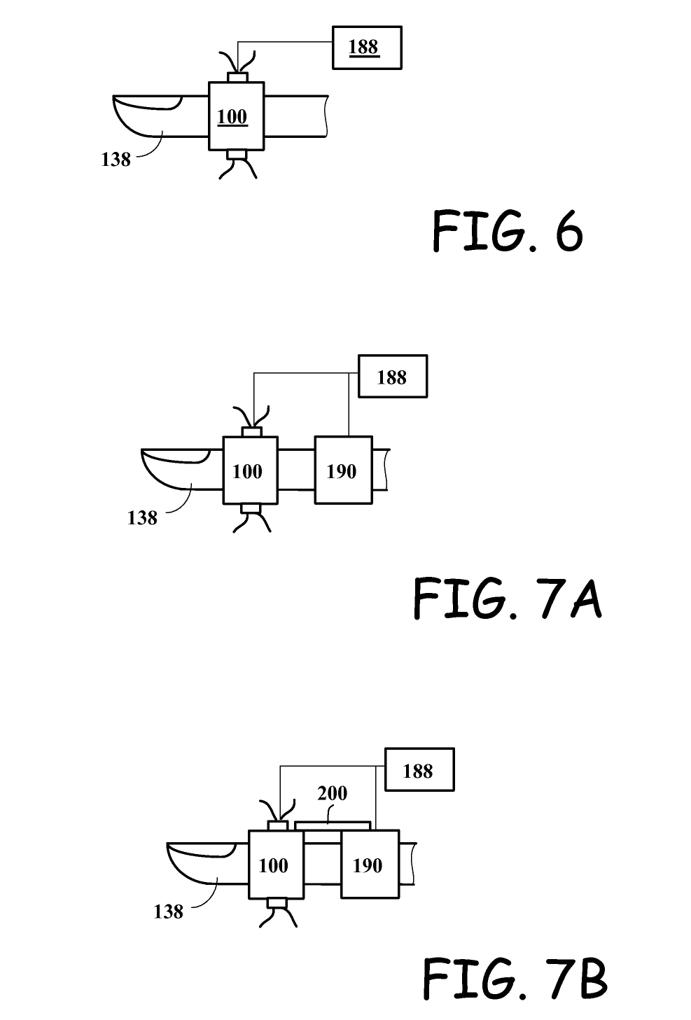

FIG. 6 is a schematic illustration of an exemplary method of application of the present probe. An object 138 such as a finger is inserted into a sleeve shaped probe 100. Both the radiation source 124 and detector 128 are connected to a controller 188 that operates them, receives measurement results, processes the results according to a certain algorithm, interprets the results of processing in terms of analyte concentration and displays the results or sends them into a displaying device. The measured analyte is a tissue and blood analyte and may be the concentration of one of hemoglobin, hematocrit, glucose, bilirubin, oxygen saturation, and other blood and tissue analytes. Upon completion of the measurements for one subject, the inner surface 114 of the probe may be cleaned by alcohol or similar fluid, where dirt and sweat left by the previous patient are removed, and the surface stickiness restored.

The measurement scheme of FIG. 6 may be sufficient for certain blood and tissue analyte determination applications for example, such as oximetry, or lower accuracy hemoglobin and the measurements of other analytes. As noted above, the optical signal measured by the detector is a weak one and typically has a poor signal to noise ratio. In order to improve the signal to noise ratio of the measured signal and make the measurement more reliable and suitable for measurements of glucose and hemoglobin, methods of blood and interstitial fluids flow intensification are used. These methods include change of temperature at the measurement point, application of pressure, including occlusion and cessation of blood flow, application of materials causing local stimulation and others.

U.S. Pat. Nos. 6,213,972, 6,400,977, 6,711,424 and 6,804,002 all presently held by the assignee of the present application, disclose different types of finger holders that in addition to regular operation, operate in pressure-release (occlusion-release) mode, under which certain pressure is applied to the finger. The pressure, which is released after a predetermined time, may include an over systolic pressure that occludes vessels and ceases the blood flow at the measurement location. The measurements may be taken through the entire pressure-release cycle, or at predetermined time intervals. As used herein the pressure-release cycle may include an occlusion-release cycle.

FIG. 7A is a schematic illustration of an exemplary method of application of the present probe and a pressure application article for measurement of physiological parameter. Object 138 is inserted into a pressure-developing article 190 such as a pneumatic cuff and into a probe 100. The controller 188 operates the radiation source, detector and the pressure sequence applied by pressure developing article 190 that enhances fluids flow and improves signal to noise ratio. Pressure developing article 190 develops a range of pressures including over-systolic pressure that occludes blood-conducting vessels and ceases blood flow. Controller 188 receives the measurement results, processes the results according to certain a algorithm, and interprets the results of processing in terms of analyte concentration. Controller 188 may include a display for visual representation of the measurement results. The display provides an instant feedback to the caregiver or person operating the apparatus. The measured analyte concentration may be one of hemoglobin, hematocrit, glucose, bilirubin, oxygen saturation, cholesterol and albumin as well as others blood or tissue analytes.

A strip 200 made of rigid material (FIG. 7B) connects between probe 100 and pressure developing article 190 and forms a unit similar to a probe. Strip 200 reduces or eliminates motion artifacts that may be produced by occasional relative movement of the probe and article caused by incidental movement of finger 138 phalanx.

In a further embodiment illustrated in FIG. 8, the pressure-developing article is incorporated into a sleeve like body of probe 210. The particular pressure-developing article is a pneumatic cuff 214 with the surface 218 of the cuff engaging the object, and is similarly a sticky surface, produced by any one of the discussed above methods. Other than pneumatic cuffs, pressure applications devices may be also used. Two mechanically locked halves of article 210 allow easy insertion of the object. Probe 210 includes one or more radiation sources 224 and at least one detector 228 arranged to measure the radiation-object interaction products. Pipe socket 230 facilitates connection to a source of compressed air for pneumatic cuff 214. Probe 210, by means of cuff 214, may operate in a pressure-release or occlusion-release cycle and the measurements may be taken through the entire pressure-release cycle, or at predetermined time intervals.

Measurement of certain analytes has a lower accuracy, and may not require application of pressure. In such cases, probe 210 will operate in a conventional mode with the sticky surface reducing or eliminating the influence of motion artifacts.

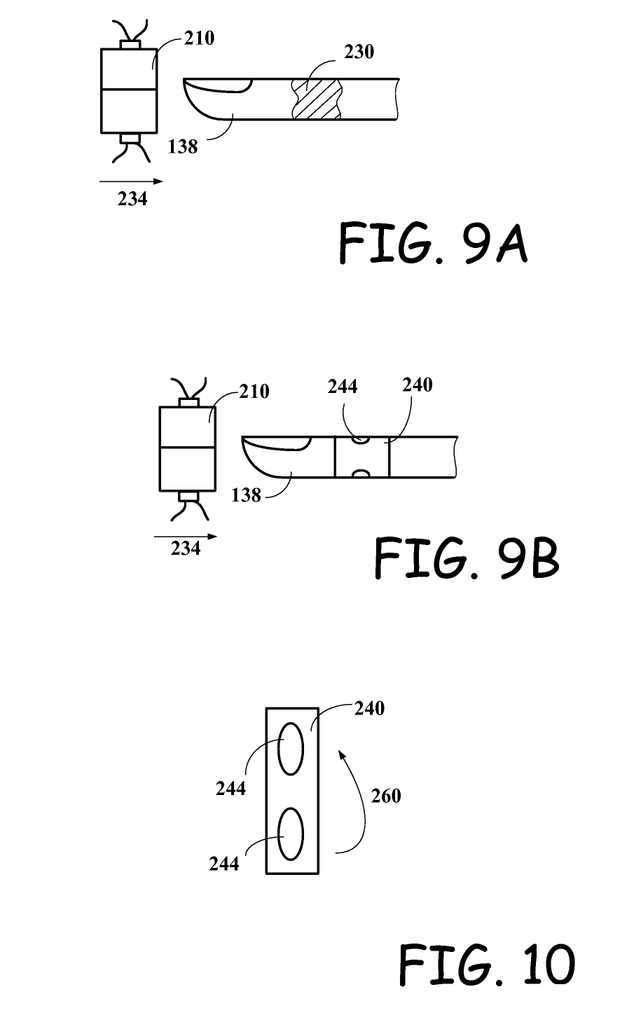

In another exemplary embodiment of the method of application of a sticky surface for measurement of physiological parameter illustrated in FIG. 9A, Repositionable Adhesive Spray 75 may be sprayed around the phalanx of the finger 138 to form a sticky coating 230 on the finger. Probe 210, 100 or any other similar probe that engages the measurement object may receive the finger and engage the sticky surface. Sticky coating 230 ensures absence of movement during the measurement of a physiological parameter, which may be a tissue or blood analyte, between the probe and the object (finger 138). Spray 75 is easy to remove by cleaning the finger by alcohol or warm water.

FIG. 9B illustrates an additional exemplary embodiment of the method of application of a sticky surface for measurement of physiological parameter. In this embodiment a double sticky transparent tape, serving as a wrapping wrapped around finger 138 may also be applied for measurement of physiological parameter. In another embodiment an opaque tape 240 having windows 244 matching the location of the radiation source and detector may be wrapped around the finger or located on an earlobe. Double sticky tape 240 greatly reduces any movement during the measurement of a physiological parameter between the probe and the object.

FIG. 10 is a schematic illustration of a double sticky wrapping for measurement of physiological parameters. Wrapping 240 has windows 244 located such that when wrapped as shown by arrow 260 around finger 138 windows 244 match the location of the radiation source and detector. Wrappings 240 may be supplied as a tape with creasing allowing for convenient detachment of a particular section of the tape, or as individual wrappings of a number of sizes matching finger sizes of different objects.

The probe disclosed and associated with it physiological parameters determination method significantly reduces and in some cases eliminates motion artifacts' influence, improves measurement reliability, and reduces the number of faulty measurements, consequently resulting in a higher-accuracy measurement with increased comfort to the subject.

Although specific embodiments have been illustrated and described herein, it will be appreciated by those of ordinary skill in the art that a variety of alternate and/or equivalent implementations may be substituted for the specific embodiments shown and described without departing from the scope of the present probe and the disclosed method. This application is intended to cover any adaptations or variations of the specific embodiments discussed herein. Therefore, it is intended that these probe and method be limited only by the claims and the equivalents thereof.

What is claimed is:

1. A probe for application to an object for non-invasive analyte measurement, said probe comprising:
a) a body with at least one sticky surface, and
b) a source of radiation and a detector built into said body.

2. The probe according to claim 1, wherein said source of radiation and detector are operable to measure a blood and tissue analyte.

3. The probe according to claim 1, wherein said body comprises one of a pressure development article, a multilayer resilient structure, or a combination of both of them.

4. The probe according to claim 1, wherein said sticky surface engages said object.

5. The probe according to claim 1, wherein said sticky surface restricts relative movements between said probe and said object.

6. The probe according to claim 1, wherein said sticky surface is one of a silicone layer having a hardness of less than 15 Shorr surface, a coating, a plurality of micron size columns, or a temporarily sprayed coating.

7. The probe according to claim 1, wherein said sticky surface is a cleanable surface.

8. The probe according to claim 1, wherein said source of radiation is a source of red and infrared radiation.

9. The probe according to claim 1, wherein the resilient structure of said body is a multilayer structure, and wherein at least one of said layers develops and supports application of pressure to said object.

10. The probe according to claim 1, wherein the resilient structure of said body is a multilayer structure, and wherein at least one of said layers has at least a section of a surface possessing sticky properties.

11. The probe according to claim 1, wherein at least one of the layers of the multilayer structure of said body is armored by one of external or internal armor.

12. The probe according to claim 1, wherein said body is a lightproof thimble-like or sleeve-like body.

13. The probe according to claim 1, wherein operation of the pressure development article of said probe includes operation in occlusion-release mode, and wherein said occlusion mode develops pressure higher than systolic pressure.

14. An apparatus for determination of a physiological parameter of a subject, said apparatus comprising:
a) a probe for receiving a measurement object, wherein the surface of said probe engaging the object is a sticky surface, and
b) a control and processing unit.

15. The apparatus according to claim 14, wherein said sticky surface restricts relative movements between said probe and the object.

16. The apparatus according to claim 14, wherein said probe is one of a resilient structure, a pressure development article, or a combination of both.

17. The apparatus according to claim 14, wherein a source of red and infrared radiation and a detector are built into said probe.

18. The apparatus according to claim 14, wherein said probe includes a pressure development article and operation of said article includes operation in occlusion-release mode.

19. The apparatus according to claim 14, wherein the pressure development article of said probe develops pressure exceeding systolic pressure.

20. The apparatus according to claim 14, wherein said physiological parameter is one of tissue or blood parameters.

21. The apparatus according to claim 14, wherein said physiological parameter is concentration of one of hemoglobin, hematocrit, glucose, bilirubin, oxygen saturation, cholesterol and albumin.

22. The apparatus according to claim 14, wherein said control and processing unit includes a display.

23. A method of a non-invasive analyte concentration measurement, said method comprising:
a) applying to a measurement object a probe with a surface engaging said object being a sticky surface; and
b) operating said probe to perform the measurement of said analyte of interest.

24. The method according to claim 23, wherein said measurement includes operation of said probe in occlude-release mode.

25. The method according to claim 23, wherein the operation of said probe in said occlude-release mode develops pressure that exceeds systolic pressure and temporarily ceases blood flow in said object.

26. The method according to claim 23, wherein operation of said probe includes operation of a source of red and infrared radiation that is built into said probe.

27. The method according to claim 23, wherein said analyte of interest is a tissue and blood analyte.

28. The method according to claim 23, wherein said analyte of interest is concentration of one of hemoglobin, hematocrit, glucose, bilirubin, oxygen saturation, cholesterol and albumin.

29. The method according to claim 23, wherein said engagement of the sticky surface with the object reduces the relative movement between said object and said structure to alleviate movement that would have a substantial effect on said concentration measurements.

30. The method according to claim 23, wherein said probe communicates the measurement data to a control and processing unit.

31. A method of reducing motion artifacts in a non-invasive analyte measurement, said method comprising:
a) applying to a measurement object a probe, wherein a surface of said probe that engages the object is a sticky surface;
b) performing the measurement of said analyte of interest, and
c) reducing said motion artifacts by restricting with the help of said sticky surface said object-probe relative movements.

32. The method according to claim 31, wherein said measurement includes operation of a source of red and infrared radiation that is built into said probe.

33. The method according to claim 31, wherein said measurement includes the application of pressure to create an occlude-release mode and wherein the pressure in said occlude-release mode exceeds systolic pressure, whereby blood flow in said object is temporarily ceased.

34. The method according to claim 31, wherein said analyte of interest is a concentration of one of hemoglobin, hematocrit, glucose, bilirubin, oxygen saturation, cholesterol and albumin.

35. A method of reducing motion artifacts in a non-invasive analyte concentration measurement, said method characterized in that a sticky surface of a probe reduces said artifacts by engaging at least a section the object of measurement.

36. A method of a non-invasive analyte concentration measurement, said method comprising:
a) engaging a surface of a probe with a surface of a measurement object and wherein at least one of said surfaces is a sticky surface; and
b) operating said probe to perform the measurement of analyte of interest.

37. The method according to claim 36, wherein said measurement includes operation of said probe in occlusion-release mode and wherein said mode develops pressure that exceeds systolic pressure and temporarily ceases blood flow in said object.

38. The method according to claim 36, wherein said measurement includes operation of a source of red and infrared radiation built into said probe.

39. The method according to claim 36, wherein said analyte of interest is a concentration of one of hemoglobin, hematocrit, glucose, bilirubin, oxygen saturation, cholesterol and albumin.

40. The method according to claim 36, wherein the engagement with said at least one sticky surface reduces the relative movement between said object and said probe in such a manner to allevate movement that may have an adverse effect on the accuracy of said analyte concentration measurements.

41. The method according to claim 36, wherein said at least one sticky surface is the surface of the object.

42. A probe for non-invasive optical analyte measurement, said probe characterized in that at least one of the surfaces of the probe engaging the measurement object is a sticky surface.
